(19) United States
(12) Patent Application Publication
(10) Pub. No.: US 2019/0314204 A1
(43) Pub. Date: Oct. 17, 2019

(54) CONTROL APPARATUS FOR WELDING AND CONTROL METHOD THEREOF

(71) Applicant: OTOS WING CO., LTD., Seoul (KR)

(72) Inventor: Moon Young HUH, Seoul (KR)

(21) Appl. No.: 15/955,025

(22) Filed: Apr. 17, 2018

(51) Int. Cl. A61F 9/06; G01J 1/44; G04F 13/02

(52) U.S. Cl. CPC A61F 9/061; G04F 13/02; G01J 1/44

(57) ABSTRACT

A welding control apparatus includes a light sensor configured to detect presence and intensity of welding light, a controller configured to count presence, intensity, and elapsed time of welding light, detected by the light sensor, and to determine welding intensity, weld time, resting time, and weld number, a memory configured to store the welding intensity, the weld time, the resting time, and the weld number, determined by the controller, a display configured to display the welding intensity, the weld time, the resting time, and the weld number, stored in the memory, a shutter driver configured to drive a shutter liquid crystal display (LCD) to vary a darkness concentration, and a setting unit configured to receive a setting value and a manipulation command, set by a user, and to transmit the setting value and the manipulation command to the controller.

### BACKGROUND OF THE INVENTION

### Field of the Invention

The present invention relates to a welding control apparatus and a control method thereof, and more particularly to a welding control apparatus and a control method thereof for determining welding intensity, weld time, resting time, and weld number using a light sensor and storing the detected information in a memory and recording, managing, and displaying a detailed welding state.

### Description of the Related Art

In general, arc welding refers to a process that is used to locally heat and dissolve metal using the fusibility of metal to join two metals and, in this regard, a worker puts on a welding helmet as one of protective devices for protection of the worker from heat, light, and gas which are generated during a welding process.

In the case of a welding helmet with a handle, it is cumbersome to release a welding helmet when in use and then to repeatedly and frequently hold and release a handle several times for every welding process.

The aforementioned welding helmet has been developed as a band type helmet and has been developed and studied to enhance work efficiency. In particular, a welding helmet is used to protect the eyes and face during an operation such as welding or cutting. In addition, the welding helmet includes an antiglare device (hereinafter, referred to as a cartridge) that is fixedly installed therein to protect user's eyes from intense harmful light generated during an operation such as welding or cutting.

In general, the cartridge blocks light with a wavelength equal to or greater than 780 nm (ib) and less than 365 nm (uv) and controls transmittance of visible light to allow a worker to perform a process while visually checking a welding position without glare.

U.S. Pat. No. 5,533,206 discloses a welding helmet including a liquid crystal display (LCD) lens that is directly positioned in front of eyes of a worker to actually function as a view window, a solar cell that absorbs light to function as an energy input unit, an electronic quick change (EQC) cartridge including an optical sensor cell that detects sparks and other intense light to function as a circuit input unit for automatically adjusting the LCD lens in a variable opacity state, and a cartridge housing positioned in a helmet to fixedly install the EQC cartridge in the helmet.

U.S. Pat. No. 6,070,264 discloses a shutter installed to allow a helmet wearer to see a welding process, an electronic controller coupled to the shutter to control light penetration shade of the shutter, an optical sensor for allowing the electronic controller to detect light emitted from the welding processing, and an electronic circuit for driving the shutter to be darker than before in response to the optical sensor that detects bright light from the welding process.

However, such a welding helmet includes a cartridge that detects intense light generated during a welding process to automatically drive an LCD lens and a shutter to be dark to protect the eyes of a worker against intense light but the cartridge is not capable of recording and managing a welding state, weld time, and weld number for each process and, thus, there is a problem in that a welding process is not effectively performed.

When welding of components, equipment, devices, and so on needs a high degree of accuracy or in an environment in which a worker has a difficulty in a welding operation, a robot performs welding and, in particular, welding of a robot is required in mass production equipment. However, in reality, it is not possible to accurately measure weld time of a robot and to accurately set a lifespan of the robot and replacement frequency of consumables and, thus, the replacement frequency of consumables is arbitrarily determined and there is a problem in that the robot malfunctions, causing production to stop.

In addition, weld time of robot welding is not accurately measured and, thus, there is a problem in terms of degraded accuracy of welding.

### CITED REFERENCE

### Patent Document

(Patent Document 1) Korean Patent Publication No. 10-1130222 (Mar. 19, 2012)

### SUMMARY OF THE INVENTION

Therefore, the present invention has been made in view of the above problems, and it is an object of the present invention to provide a welding control apparatus and a control method thereof for determining welding intensity, weld time, resting time, and weld number using a light sensor and storing the detected information in a memory and recording, managing, and displaying a detailed welding state.

It is another object of the present invention to provide a welding control apparatus and a control method thereof for accurately measuring weld time of a robot to set a lifespan of the robot and replacement frequency of consumables and, also, accurately measuring weld time of robot welding to effectively perform management.

In accordance with the present invention, the above and other objects can be accomplished by the provision of a welding control apparatus including a light sensor configured to detect presence and intensity of welding light, a controller configured to count presence, intensity, and elapsed time of welding light, detected by the light sensor, and to determine welding intensity, weld time, resting time, and weld number, a memory configured to store the welding intensity, the weld time, the resting time, and the weld number, determined by the controller, a display configured to display the welding intensity, the weld time, the resting time, and the weld number, stored in the memory, a shutter driver configured to drive a shutter liquid crystal display (LCD) to vary a darkness concentration according to control of the controller, and a setting unit configured to receive a setting value and a manipulation command, set by a user, and to transmit the setting value and the manipulation command to the controller.

The controller may recognize a welding operation to be started upon detecting first welding light from the light sensor and, then, counts an elapsed time up to a time point when welding light is not detected to determine the weld time, the controller may recognize the first welding light not to be detected from the light sensor and, then, counts an elapsed time up to a time point when the welding light is detected to determine the resting time, the controller may count a number of times that the welding light is detected from the light sensor to determine the weld number, and the controller may detect intensity of the welding light from the light sensor, may count the weld time, and may compare the weld time with a predetermined reference value to determine the welding intensity depending on how long the weld time is maintained at specific intensity.

The welding control apparatus may further include an image detector configured to detect an image in a welding state, wherein the memory may store the image in the welding state, detected by the image detector.

The welding control apparatus may further include a general welding robot, wherein the controller may measure welding operation time of the welding robot and may display the welding operation time on the display in real time.

In accordance with another aspect of the present invention, there is provided a welding control method including detecting a welding signal by detecting presence and intensity of welding light from a light sensor, determining effective welding by detecting presence, intensity, and an elapsed time of the welding light detected from the light sensor and comparing detected information with a reference value, by a controller, counting the presence, the intensity, and the elapsed time of the welding light detected from the light sensor to determine welding intensity, weld time, resting time, and weld number, and storing the welding intensity, the weld time, the resting time, and the weld number in a memory, by the controller, and detecting an image in a welding state by an image detector according to input of a setting unit and control of the controller and storing the image in the welding state, detected by the image detector, in a memory.

The counting of the presence, the intensity, and the elapsed time may include recognizing a welding operation to be started upon detecting first welding light from the light sensor and, then, counting an elapsed time up to a time point when welding light is not detected to determine the weld time, recognizing the first welding light not to be detected from the light sensor and, then, counting an elapsed time up to a time point when the welding light is detected to determine the resting time, counting a number of times that the welding light is detected from the light sensor to determine the weld number, and detecting intensity of the welding light from the light sensor, counting the weld time, and comparing the weld time with a predetermined reference value to determine the welding intensity depending on how long the weld time is maintained at specific intensity.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a perspective view showing an outer appearance of a welding helmet according to the present invention;

FIG. 2 is a diagram showing a cartridge of a welding helmet according to the present invention;

FIG. 3 is a block diagram showing a welding helmet control device according to the present invention; and

FIG. 4 is a control flowchart of a welding helmet control method according to the present invention.

### DETAILED DESCRIPTION OF THE INVENTION

As the invention allows for various changes and numerous embodiments, particular embodiments will be illustrated in the drawings and described in detail in the written description. In the description of the present invention, certain detailed explanations of the related art are omitted when it is deemed that they may unnecessarily obscure the essence of the invention.

Reference will now be made in detail to the exemplary embodiments of the present invention with reference to the accompanying drawings.

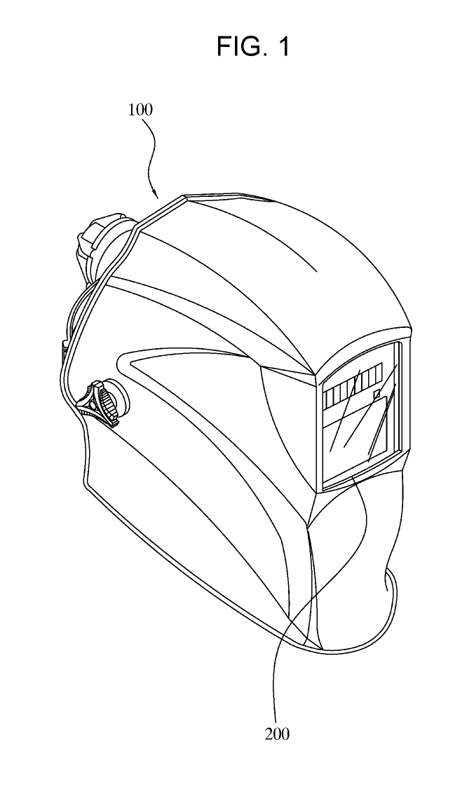

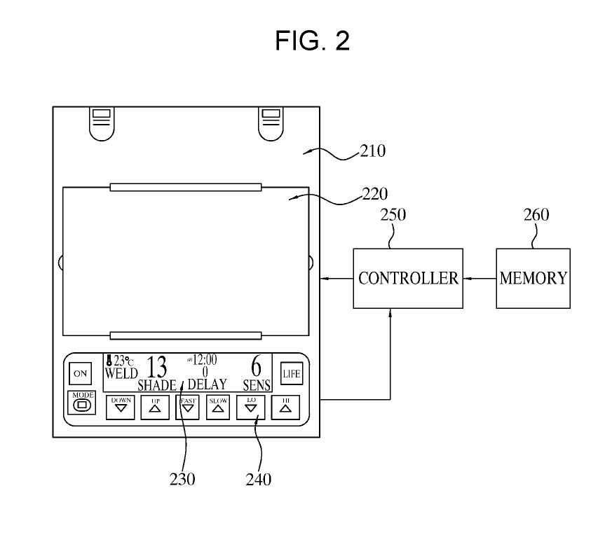

FIG. 1 is a perspective view showing an outer appearance of a welding helmet 100 according to the present invention. FIG. 2 is a diagram showing a cartridge 200 of the welding helmet 100 according to the present invention.

As illustrated, the welding helmet 100 according to the present invention may include the cartridge 200 and the cartridge 200 may include a main body 210, a shutter liquid crystal display (LCD) 220, a display 230, and a setting unit 240.

The welding helmet 100 may be formed to protect and cover a face of a worker and may include the cartridge 200 installed on a front surface portion of the welding helmet 100.

The welding helmet 100 may be formed of a light material such as an incombustible plastic.

The main body 210 of the cartridge 200 may configure an outer appearance of the cartridge 200 and may include a controller 250 including a general central processing unit (CPU) and a memory 260, which are installed in the main body 210.

Darkness concentration of the shutter LCD 220 may be set according to a user manipulation command or control of the controller 250 and the shutter LCD 220 may be driven with variable darkness concentration according to driving of a shutter driver 221.

A worker may wear the welding helmet 100, may cover his or her face with the cartridge 200 at the front surface portion and, then, may perform welding or cutting-off with appropriate brightness through an operation of the shutter LCD 220.

The display 230 may display a user input state, a device operation state, or the like.

The setting unit 240 may receive a setting value and a manipulation command set by a user and may transmit the received information to the controller 250.

The controller 250 may control an overall operation state of a device and the memory 260 may store a data value based on a setting value and an operation state.

A configuration and operation of the welding helmet configured as described above according to the present invention will be described in more detail.

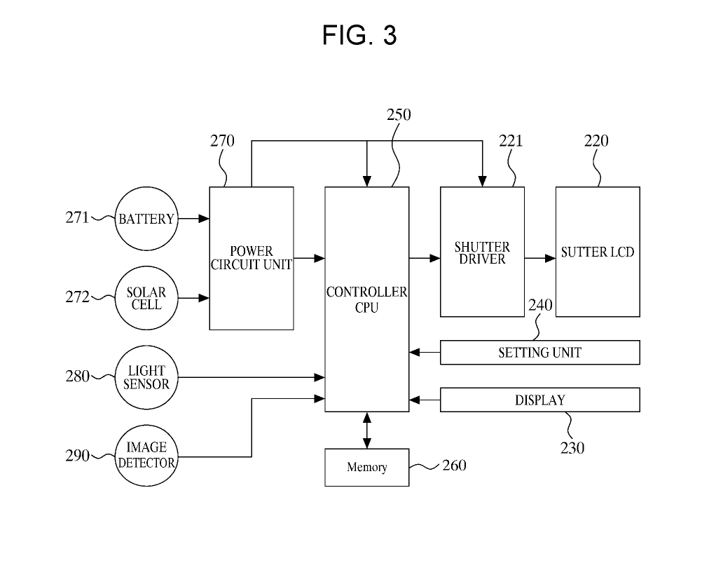

FIG. 3 is a block diagram showing a welding helmet control device according to the present invention.

As illustrated, the welding helmet control device according to the present invention may include the shutter LCD 220, the shutter driver 221, the display 230, the setting unit 240, the controller 250, the memory 260, a power circuit unit 270, a battery 271, a solar cell 272, a light sensor 280, and an image detector 290.

The power circuit unit 270 may supply power collected from the battery 271 or the solar cell 272 to each unit.

The light sensor 280 may detect presence and intensity of welding light.

The image detector 290 may detect an image of a welding state.

The controller 250 may count presence, intensity, and elapsed time of welding light, detected by the light sensor 280, and may determine welding intensity, weld time, resting time, and weld number.

The memory 260 may store the welding intensity, the weld time, the resting time, and the weld number, determined by the controller 250. The memory 260 may store the image of the welding state, detected by the image detector 290.

The display 230 may display the welding intensity, the weld time, the resting time, and the weld number, stored in the memory 260.

The shutter driver 221 may drive the shutter LCD 220 to vary a darkness concentration.

The setting unit 240 may receive the setting value and the manipulation command, set by the user, for example, a darkness concentration and an operating time of the shutter LCD 220 and may transmit the received information to the controller 250.

In this case, a procedure of determining weld time by the controller 250 according to the present invention is now described. First, when the light sensor 280 detects first welding light, a welding operation may be recognized to be started. Then, an elapsed time may be counted up to a time point when welding light is not detected to determine the weld time.

A procedure of determining resting time according to the present invention is now described. First welding light may be recognized not to be detected from the light sensor 280 and, then, an elapsed time may be counted up to a time point when welding light is detected to determine the resting time.

A procedure of determining weld number according to the present invention is now described. A number of times that welding light is detected from the light sensor 280 may be counted to determine the weld number.

In addition, the procedure of determining the weld number is now described. Intensity of welding light from the light sensor 280 may be detected and the weld time may be counted. The weld time may be compared with a predetermined reference value to determine welding intensity depending on how long weld time is maintained at specific intensity.

The welding state may be detected by the image detector 290, stored in the memory 260 and, then, displayed on the display 230 according to user selection.

The present invention may further include a general welding robot and the controller 250 may measure welding operation time of the welding robot and may display the information on the display 230 in real time and, thus, a user may recognize the welding operation time of the welding robot in real time.

As is well known, a welding robot is used in spot welding and arc welding and, in this regard, spot welding is a welding method of strongly pressing a plurality of thin plates using electrodes at opposite sides and allowing current to flow therethrough to attach the thin plates point by point. A robot used in spot welding is a simplest robot including a spot welding gun installed at a wrist of an arm and has a degree of freedom of 4 to 5 and is used on production lines in industries such as the automobile industry. Arc welding is a commonest welding method using high temperature generated via arc discharge. A robot used in arc welding has a function of visualizing a torch (a flame outlet) by a sensor and correcting a position, etc. of the torch according to the characteristics of arc welding. In the future, it is expected that the function and accuracy of robots will be enhanced and the robots will be employed in all production places.

A welding robot applied to the present invention is nearly the same as a well-known general robot for replacing general welding with automatic welding and, thus, a detailed description of a configuration and operation of the welding robot is omitted herein.

Accordingly, the present invention may determine welding intensity, weld time, resting time, and weld number using a light sensor, may store the detected information in a memory, and may record, manage, and display a detailed welding state to more effectively perform welding.

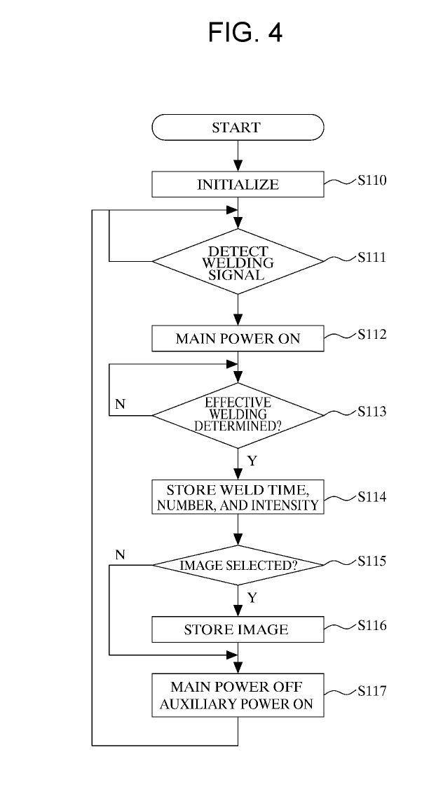

FIG. 4 is a control flowchart of a welding helmet control method according to the present invention.

As illustrated, the welding helmet control method according to the present invention may include initialization (S110), detecting a welding signal (S111), determining effective welding (S112 to S113), determining and storing weld time, weld number, and welding intensity (S114), selecting and storing an image (S115 to S116), and selecting and using main power or auxiliary power (S117).

In the initialization (S110), all settings values of a system may be initialized and the system may enter an action preparation state.

In this case, the setting unit 240 may receive a setting value and a manipulation command set by a user, for example, a darkness concentration and an operating time of the shutter LCD 220 and may transmit the received information to the controller 250.

In the detecting of the welding signal (S111), the light sensor 280 may detect presence and intensity of welding light to detect the welding signal.

In the determining of the effective welding (S112 to S113), the controller 250 may detect presence, intensity, and elapsed time of welding light, detected by the light sensor 280, and may compare the detected information with a reference value to determine effective welding.

In the determining and storing of the weld time, the weld number, and welding intensity (S114), the controller 250 may count presence, intensity, and elapsed time of welding light, detected by the light sensor 280, and may determine welding intensity, weld time, resting time, and weld number.

In this case, in a procedure of determining the weld time by the controller 250 according to the present invention, when the light sensor 280 detects first welding light, a welding operation may be recognized to be started. Then, an elapsed time may be counted up to a time point when welding light is not detected to determine the weld time.

In a procedure of determining resting time according to the present invention, first welding light may be recognized not to be detected from the light sensor 280 and, then, an elapsed time may be counted up to a time point when welding light is detected to determine the resting time.

In a procedure of determining weld number according to the present invention, a number of times that welding light is detected from the light sensor 280 may be counted to determine the weld number.

In addition, in the procedure of determining the weld number according to the present invention, intensity of welding light from the light sensor 280 may be detected and the weld time may be counted. The weld time may be compared with a predetermined reference value to determine welding intensity depending on how long weld time is maintained at specific intensity.

In the selecting and storing of the image (S115 to S116), the image detector 290 may detect an image of a welding state according to input of the setting unit 240 and control of the controller 250 and the memory 260 may store the image of the welding state, detected by the image detector 290.

When the main power is turned off, the auxiliary power may be turned on to supply power.

Accordingly, the present invention may determine welding intensity, weld time, resting time, and weld number using a light sensor, may store the detected information in a memory, and may record, manage, and display a detailed welding state to more effectively perform welding.

As is apparent from the above description, the present invention provides a welding helmet control device and a control method thereof, for determining welding intensity, weld time, resting time, and weld number using a light sensor and storing the detected information in a memory and recording, managing, and displaying a detailed welding state to more effectively perform welding.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A welding control apparatus comprising:
a light sensor configured to detect presence and intensity of welding light;
a controller configured to count presence, intensity, and elapsed time of welding light, detected by the light sensor, and to determine welding intensity, weld time, resting time, and weld number;
a memory configured to store the welding intensity, the weld time, the resting time, and the weld number, determined by the controller;
a display configured to display the welding intensity, the weld time, the resting time, and the weld number, stored in the memory;
a shutter driver configured to drive a shutter liquid crystal display (LCD) to vary a darkness concentration according to control of the controller; and
a setting unit configured to receive a setting value and a manipulation command, set by a user, and to transmit the setting value and the manipulation command to the controller.

2. The welding control apparatus according to claim 1, wherein the controller recognizes a welding operation to be started upon detecting first welding light from the light sensor and, then, counts an elapsed time up to a time point when welding light is not detected to determine the weld time;
wherein the controller recognizes the first welding light not to be detected from the light sensor and, then, counts an elapsed time up to a time point when the welding light is detected to determine the resting time;
wherein the controller counts a number of times that the welding light is detected from the light sensor to determine the weld number; and
wherein the controller detects intensity of the welding light from the light sensor, counts the weld time, and compares the weld time with a predetermined reference value to determine the welding intensity depending on how long the weld time is maintained at specific intensity.

3. The welding control apparatus according to claim 1, further comprising an image detector configured to detect an image in a welding state,
wherein the memory stores the image in the welding state, detected by the image detector.

4. The welding control apparatus according to claim 1, wherein the welding control apparatus is further included in a welding helmet; and
wherein the controller measures welding operation time of the welding helmet and displays the welding operation time on the display in real time.

5. The welding control apparatus according to claim 1, further comprising a welding robot,
wherein the controller measures welding operation time of the welding robot and displays the welding operation time on the display in real time.

6. A welding control method comprising, the method comprising:
detecting a welding signal by detecting presence and intensity of welding light from a light sensor;
determining effective welding by detecting presence, intensity, and an elapsed time of the welding light detected from the light sensor and comparing detected information with a reference value, by a controller;
counting the presence, the intensity, and the elapsed time of the welding light detected from the light sensor to determine welding intensity, weld time, resting time, and weld number, and storing the welding intensity, the weld time, the resting time, and the weld number in a memory, by the controller; and
detecting an image in a welding state by an image detector according to input of a setting unit and control of the controller and storing the image in the welding state, detected by the image detector, in a memory.

7. The method of claim 6, wherein the counting of the presence, the intensity, and the elapsed time comprises:
recognizing a welding operation to be started upon detecting first welding light from the light sensor and, then, counting an elapsed time up to a time point when welding light is not detected to determine the weld time;
recognizing the first welding light not to be detected from the light sensor and, then, counting an elapsed time up to a time point when the welding light is detected to determine the resting time;
counting a number of times that the welding light is detected from the light sensor to determine the weld number; and
detecting intensity of the welding light from the light sensor, counting the weld time, and comparing the weld time with a predetermined reference value to determine the welding intensity depending on how long the weld time is maintained at specific intensity.
